(19) United States
(12) Patent Application Publication
(10) Pub. No.: US 2026/0315427 A1
(43) Pub. Date: Oct. 15, 2026

(54) TLR4 INHIBITORS FOR CANCER TREATMENT

(71) Applicant: Thomas Jefferson University, Philadelphia, PA (US)

(72) Inventor: Neda Nikbakht, Philadelphia, PA (US)

(21) Appl. No.: 19/113,781

(22) PCT Filed: Sep. 21, 2023
(86) PCT No.: PCT/US23/33388

§ 371 Date: Mar. 20, 2025

Related U.S. Application Data
(60) Provisional application No. 63/409,011, filed on Sep. 22, 2022.

(51) Int. Cl. A61K 31/216; A61K 45/06; A61P 35/00; A61P 37/04

(52) U.S. Cl. CPC A61K 31/216; A61K 45/06; A61P 35/00; A61P 37/04

(57) ABSTRACT

The present disclosure relates to methods and compositions useful for treating cancer in a subject in need thereof. Said methods and compositions comprising inhibitors of toll-like receptor 4 (TLR4) signaling.

### CROSS-REFERENCE TO RELATED APPLICATION

The present application claims priority under 32 U.S.C. § 119(e) to U.S. Provisional Patent Application No. 63/409,011, filed Sep. 22, 2022, which is hereby incorporated by reference in its entirety herein.

### STATEMENT REGARDING FEDERALLY SPONSORED RESEARCH OR

### DEVELOPMENT

This invention was made with government support under CA252818-01 awarded by the National Institutes of Health. The government has certain rights in the invention.

### BACKGROUND

Cutaneous T cell lymphomas are non-Hodgkin lymphomas that are characterized by clonal proliferations of T-lymphocytes and typically involve the skin and some mucosal tissues. While typically characterized as a non-aggressive disease, they can develop into rapidly progressive subtypes.

Toll-Like Receptor 4 or TLR4 is a transmembrane receptor protein of the toll-like receptor family. Like other toll-like family members, TLR4 is primarily a pattern recognition receptor (PRR) which senses the presence of molecular ligands directly or indirectly associated with pathogen infection. In the case of TLR4, these ligands include lipopolysaccharide, a cell wall component of many Gram-negative bacteria, as well as endogenous proteins which are “danger signals” released by on-going infections including low-density lipoproteins, beta-defensins, health shock proteins, and palmitic acid. While normally expressed by myeloid-linage cells, including macrophages and dendritic cells, high expression levels of TLR4 in lymphomas, including cutaneous T cell lymphoma, are accompanied by high expression of other immunosuppressive checkpoint inhibitor molecules and, ultimately, with treatment resistance and worse prognosis.

There is thus a need in the art for therapeutic methods and compositions suitable for the treatment of cancers, including cutaneous T-cell lymphoma, which leverage inhibition of TLR4 signaling. The present invention addresses this need.

### SUMMARY

As described herein, the current disclosure relates to methods and compositions useful for treating cancer in a subject in need thereof. Said methods and compositions comprising inhibitors of toll-like receptor 4 (TLR4) signaling.

As such, in one aspect, disclosed herein is a method for treating cancer in a subject in need thereof, comprising administering to the subject an effective amount of an inhibitor of Toll-Like Receptor 4 (TLR4), thereby treating the cancer;

wherein treatment with the TLR4 inhibitor induces a pro-inflammatory phenotype in at least one tumor-associated immune cell.

In some embodiments, the TLR4 inhibitor is TAK-242.

In some embodiments, the tumor-associated immune cell is a monocyte selected from the group consisting of a macrophage, a dendritic cell, or any combination thereof.

In some embodiments, the tumor-associated immune cell is a macrophage.

In some embodiments, the macrophage is a M1 macrophage.

In some embodiments, the pro-inflammatory phenotype is a change in the ratio of M1 to M2 macrophages.

In some embodiments, the cancer is a lymphoma.

In some embodiments, the lymphoma is cutaneous T-cell lymphoma (CTCL).

In some embodiments, the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

In some embodiments, the TLR4 inhibitor is delivered systemically.

In some embodiments, the TLR4 inhibitor is delivered topically.

In some embodiments, the method of the above aspect or the method of any aspect or embodiment disclosed herein further comprises administering to the subject at least one other therapeutic agent concurrent with, prior to, or subsequent to the TLR4 inhibitor.

In some embodiments, the therapeutic agent is selected from the group consisting of a systemic chemotherapy, a topical chemotherapy, an immunotherapy, an antibody-drug conjugate (ADC), an antibody, an immunotoxin, an interferon, and any combination thereof.

In another aspect, disclosed herein is a composition for treating cancer, comprising an effective amount of a TLR4 inhibitor and a pharmaceutically acceptable carrier or excipient.

In some embodiments, the TLR4 inhibitor is TAK-242.

In some embodiments, the cancer is a lymphoma.

In some embodiments, the lymphoma is cutaneous T-cell lymphoma.

In some embodiments, the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

### BRIEF DESCRIPTION OF THE FIGURES

The drawings illustrate generally, by way of example, but not by way of limitation, various embodiments of the present application.

FIGS. 1A-1F illustrate the impact of loss of TLR4 on tumor-infiltrating macrophages. (FIG. 1A) 1.2×10⁶ MBL2 cells were injected s.c. into flanks of C57BL/6 and TLR4^{-/-} recipients (5 mice/grp.) and tumor volumes monitored overtime. Shown are the mean and standard error for each group. (B-F) Tumors were generated as described in A in mice of indicated genotypes and mice were euthanized when tumor volumes in wild-type reached 1000-1500 mm. Single cell suspensions were obtained by enzymatic digestion of tumors and skins, then stained by indicated antibodies and analyzed using flow cytometry. (B) Gating strategy to define M2 macrophages in tumors of indicated genotypes as CD11b⁺F4/80⁺CD206⁺. (C-F) M1:M2 macrophage ratios compared between skin and tumors of tumor bearing mice of each indicated genotype.

FIG. 2 illustrates the effect of TLR4 inhibitor treatment on tumor growth in vivo. 2×10⁶ MBL2 cells were injected s.c. into the flanks of C57BL/6 mice. When tumors reached an average volume of 85 mm, mice were allocated to vehicle (n=5) or the TLR4 inhibitor TAK-242 (n=5) treatment groups. All mice were treated daily with intraperitoneal injections of vehicle (95% saline 5% DMSO) or 3 mg/kg of TAK-242. Tumor volumes were monitored three times per week. Tumor sizes are shown as average and SEM per group.

FIG. 3 is a diagram of the molecular structure of TAK-242.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise.

Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range and includes the exact stated value or range.

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequence or a partial nucleotide sequence encoding a protein that elicits an adaptive immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The term “immune cell,” as used herein refers to any cell involved in the mounting of an immune response. Such cells include, but are not limited to, T cells, B cells, NK cells, antigen-presenting cells (e.g., dendritic cells and macrophages), monocytes, neutrophils, eosinophils, basophils, and the like.

The term “local delivery,” as used herein, refers to delivery of an active agent or therapeutic agent such as an inhibitor directly to a target site within an organism. For example, an agent can be locally delivered by direct injection into a disease site such as a tumor or other target site such as a site of inflammation or a target organ such as the liver, heart, pancreas, kidney, and the like.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a proteins or peptide's sequence.

Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “room temperature” as used herein refers to a temperature of about 15 °C to 28 °C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt. % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In one embodiment, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition.

The term “therapeutic protein” includes entire proteins or peptides and can also refer to therapeutically active fragments thereof. It can also include therapeutically active variants of a protein. Exemplary therapeutic proteins include, but are not limited to, an analgesic protein, an anti-inflammatory protein, an anti-proliferative protein, a proapoptotic protein, an anti-angiogenic protein, a cytotoxic protein, a cytostatic protein, a cytokine, a chemokine, a growth factor, a wound healing protein, a pharmaceutical protein, or a pro-drug activating protein.

Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

### Description

The invention of the present disclosure is based on the unexpected finding that ablation of TLR4 signaling, either by genetic knockout or by the use of an inhibitor, can significantly slow the growth of lymphomas, including cutaneous T-cell lymphomas (CTCLs) in vivo and that this delay was associated with the shift in phenotype of tumor-resident monocytes, including macrophages, from one associated with immunosuppression and tolerance to one associated with pro-inflammatory responses, suggesting the enhancement of endogenous anti-tumor immune responses. This strategy of inhibiting TLR4 signaling to treat cancers such as CTCL is novel compared to other TLR4-associated treatment strategies known in the art, which encompass TLR4 activation. As such, in some aspects, the current invention provides methods and compositions of treating cancers (e.g., CTCLs) through the inhibition of TLR4 signaling. Also provided are compositions useful for treating cancers (e.g., CTCLs) comprising TLR4 inhibitors.

### Cutaneous Lymphomas

Cutaneous T cell lymphomas (CTCL) are a relatively heterogenous group of distinct T cell lymphoproliferative disorders that typically involve the skin and certain mucosal tissues.

Cutaneous lymphomas typically occur extra nodal and are classified as non-Hodgkin lymphomas. The incidence of CTCL has been increasing and is currently 6.4 cases per million persons, based on recent epidemiology data, with the highest incidence rates being reported among males and African Americans. The incidence of CTCL increases significantly with age, with a median age at diagnosis around 55 and a four-fold increase in incidence in individuals over 70 years of age.

The majority (roughly 75%) of CTCL are classified as Mycosis fungoides (MF) or Sezary syndrome (SS), which are distinct in clinical presentation and apparent cell of origin. MF or Alibert-Bazin syndrome is the most common form of CTCL. Mycosis fungoides typically develops slowly and proceeds through several phases from a scaly rash to a thin, patchy rash, to distinct tumors. Sezary syndrome is a leukemic form of CTCL in which aberrant T cells can be observed in the blood as well as in cutaneous tissues. Sezary syndrome can often, but not always represent a phase of disease that started as Mycosis fungoides before developing into a systemic form.

In certain embodiments, the current invention provides methods and compositions useful for treating cancers, especially lymphomas such as CTCL in subjects in need thereof. It is contemplated that the methods and compositions of the current invention can be used to treat all forms of CTCL, including Mycosis fungoides and Sezary syndrome, but also other forms of CTCL including, but not limited to cutaneous CD30⁺ lymphoproliferative disorders, primary cutaneous gamma-delta T-cell lymphomas, anaplastic large cell lymphoma, lymphomatoid papulosis, T-cell lymphomas, primary cutaneous acral CD8⁺ T-cell lymphoma, primary cutaneous CD4⁺ small/medium T-cell lymphoproliferative disorder, Epstein-Barr virus (EBV) positive mucocutaneous ulcers, and the like.

Treatment of CTCL is typically adapted for the severity or stage of each individual patient with therapeutic agents which are biological signaling modulators being preferred to systemic chemotherapies for early-stage cases. Such biological signaling modulators include, but are not limited to, Bexarotene, a retinoid that selectively activates retinoid X receptors (RXRs) and interferon-alpha. Another class of therapeutics commonly used in the treatment of CTCL are histone deacetylase inhibitors (HDACs) including but not limited to vorinostat and romidepsin among others. More advanced cases are typically treated with combinations of radiation and commonly used systemic chemotherapy drugs including cyclophosphamide, doxorubicin, etoposide, and vincristine, among others.

The increasing availability of antibody-based therapeutics has also affected the treatment of CTCL, with antibodies targeting cell-surface molecules such as CD52 (alemtuzumab), CCR4 (mogamulizumab), and CD30 (brentuximab) demonstrating efficacy in both MF and SS patients. Additional antibody-based therapies have also demonstrated efficacy, including Resimmune, an immunotoxin comprising diphtheria toxin fused to anti-CD3 single-chain antibodies. Combination therapy with immune checkpoint inhibiting antibodies, such as anti-PD-1, anti-PD-L1/2, anti-CTLA4 and the like have demonstrated early promise in increasing patient response rate

In certain embodiments, the methods provided by the current invention can combine TLR4 inhibitors with one or more additional therapeutic agents useful for the treatment of CTCL. It is contemplated that such additional therapeutic agents can include any combination of the agents listed here, or any additional therapeutic agents including small molecule inhibitors, biologic function modulators, peptides, antibodies or antigen-binding fragments thereof, antibody-drug conjugates, chimeric antigen T cells, bispecific antibodies, and the like. It is understood that the skilled artisan would be able to select one or more additional therapeutic agents for use with the methods and compositions of the invention based on a particular patient's diagnosis and prognosis.

### Toll-like Receptors and TLR4

Toll-like receptors (TLRs) are type 1 transmembrane proteins comprising an extracellular ligand-binding domain and an intracellular signaling domain. TLRs primarily function as pattern recognition receptors (PRRs) which bind to endogenous and pathogen-associated molecules and act to detect the presence of pathogens. As such, TLRs function as critical initiators of immune responses.

Toll-Like Receptor 4 or TLR4 is a transmembrane receptor protein of the toll-like receptor family. Like other toll-like family members, TLR4 is primarily a pattern recognition receptor (PRR) which senses the presence of molecular ligands directly or indirectly associated with pathogen infection. In the case of TLR4, these ligands include lipopolysaccharide, a cell wall component of many Gram-negative bacteria, as well as endogenous proteins which are “danger signals” released by on-going infections including low-density lipoproteins, beta-defensins, health shock proteins, and palmitic acid. TLR4 is typically expressed by myeloid-derived immune cells, especially monocytes, macrophages, granulocytes, and immature dendritic cells. High expression levels of TLR4 have also been found associated with certain types of cancers, especially lymphomas, including cutaneous T cell lymphoma, are accompanied by high expression of other immunosuppressive checkpoint inhibitor molecules and, ultimately, with treatment resistance and worse prognosis.

Because TLR4 activation is associated with the early phases of immune responses, TLR4-targeting therapies have typically involved agonists and/or other agents that activate TLR4 signaling for the purpose of enhancing immune activation and priming. In certain embodiments of the current invention, however, a treatment benefit is derived from the inhibition of TLR4 signaling. This inhibition results in reduced or inhibited growth of lymphoma cells and is associated with phenotype changes to tumor-associated macrophages from M2-type to M1-type.

### TLR4 Inhibitors

In certain embodiments, the current invention provides methods and compositions comprising inhibitors of TLR4 signaling and function. A number of different TLR4 inhibitor or antagonists are known in the art, and include, but are not limited to, competitive inhibitors such as FP7-like glycolipids, trehalose derivatives (LAM), IAXO, Calixarenes, and unsaturated cardiolipins among others; down-regulators of TLR4 expression such as alpinetin; disruptors of TLR4/MD2 complex formation including ferulic acid; and non-competitive inhibitors. One such non-competitive inhibitor is TAK-242. TAK-242 is a small molecule that binds to the intracellular domain of TLR4 and inhibits the activation of downstream NFκB, IRF-3, and MAPK signaling pathways.

In certain embodiments, the TLR4 inhibitor of the current invention is TAK-242. It is also contemplated that any TLR4 inhibitor can be used in the methods and compositions of the current invention, and that the skilled artisan would be able to select an appropriate inhibitor.

### Tumor-Associated Macrophages

Macrophages are professional antigen presenting and phagocytic cells of the innate immune system and are a member of the monocyte family of myeloid-derived immune cells.

Together with neutrophils, macrophages act as first responder cells to infections and injury, where they act to recognize, phagocytose, and degrade cellular debris, bacterial cells, and viral particles. Macrophages can also prime adaptive T cell responses by presenting antigens which they phagocytose and degrade to T cells and deliver co-stimulatory signals via expressing membrane-bound signaling molecules and secreting soluble signaling molecules (e.g., cytokines and chemokines). These signaling molecules also mediate the initiation of inflammation, which in turn recruit other immune cells to inflammation sites.

Macrophages are potent effectors of the innate immune system and are capable of at least three distinct anti-tumor functions: phagocytosis, cellular cytotoxicity, and antigen presentation to prime and orchestrate an adaptive T-cell and B-cell mediated immune response. While T cells require antigen-dependent activation via the T cell receptor, macrophages can be activated in a variety of ways. Direct macrophage activation is antigen-independent, relying on mechanisms such as pathogen associated molecular pattern recognition by Toll-like receptors (TLRs).

Immune-complex mediated activation is antigen dependent but requires the presence of antigen-specific antibodies and absence of the inhibitory CD47-SIRPα interaction.

Macrophages occur in almost all tissues and have functional abilities in addition to the mediation of immune responses to pathogens and initiation of inflammation responses. Macrophages also function in the maintenance of tissue homeostasis, as well as in the repair and remodeling of tissues. These functions are known to be associated with many diseases, including metabolic and autoimmune diseases, cancers, infections, obesity, and fibrosis.

Recent studies suggest that macrophages are abundant in the tumor microenvironment of numerous cancers where they play a critical role in the regulating the tumor microenvironment, specifically in matrix remodeling, angiogenesis, metastasis, and tumor progression in addition to their role in mediating inflammation and immunity. Tumor-resident, or tumor-associated macrophages can adopt one of two broad phenotypes: a classically activated, proinflammatory phenotype (M1) characterized by the secretion of immune-activating factors including TNF-α, IL-17A, IL-1, and IL-6 or an alternatively activated, anti-inflammatory phenotype (M2) characterized by the secretion immunosuppressive cytokines including IL-4, IL-10, IL-13, and TGFβ. In general, M1 macrophages, due to their inflammatory nature, are better able to support and encourage anti-tumor immune responses, while M2 macrophages are associated with immune tolerance and suppression.

Tumor-associated macrophages have been shown to be re-programmable by the tumor microenvironment to become key immunosuppressive players in the microenvironment.

Therefore, the ability to alter the signals received by tumor-resident macrophages to encourage the development of a pro-inflammatory phenotype would help support and augment both endogenous anti-tumor immune responses as well as immunotherapies such as adoptive cell therapies (e.g., CAR-T cells and the like).

In certain embodiments, the current disclosure provides methods for treating cancer by inducing pro-inflammatory phenotypes in tumor-associated immune cells. In certain embodiments, this induction is accomplished by administering an inhibitor of TLR4 signaling.

In certain embodiments, the tumor-associated immune cell is a monocyte selected from the group consisting of a macrophage, a dendritic cell, or any combination thereof. In certain embodiments, the tumor-associated immune cell is a macrophage, specifically an M1 macrophage. In certain embodiments, the TLR4 inhibitor promotes a change or increase in the ratio of M1 to M2 macrophages in a subject. In this way, the balance of signals provided by the macrophages is also changed to be more pro-inflammatory and to support efficacious anti-tumor immune responses.

### Methods

In another aspect, the present disclosure provides a method of treating, preventing, and/or ameliorating cancer in a subject, the method comprising administering to the subject an effective amount of a composition comprising an inhibitor of Toll-Like Receptor 4 (TLR4), thereby treating the cancer. In certain embodiments, the TLR4 inhibitor induces a pro-inflammatory phenotype in at least one tumor-associated immune cell. In certain embodiments, the tumor-associated immune cell is a monocyte. A number of monocyte populations are known to associate with tumor tissue and can affect growth or suppression of tumor growth, including but not limited to macrophages, dendritic cells, monocyte precursors such as myeloid derived suppressor cells (MDSC), or any combination thereof. In certain embodiments, the TLR4 inhibitor induces a pro-inflammatory M1 phenotype in tumor associated macrophages, which is associated with inhibition of tumor growth.

In certain embodiments, the TLR4 inhibitor is a small molecule non-competitive inhibitor. In certain embodiments, the TLR4 inhibitor is TAK-242. In certain embodiments, the TLR4 inhibitor is delivered systemically. In certain embodiments, the TLR4 inhibitor is delivered topically.

In certain embodiments, the cancer is a lymphoma. In certain embodiments, the lymphoma is cutaneous T-cell lymphoma (CTCL). CTCL is a heterogenous collection of related lymphomas. Therefore, in certain embodiments, cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

In certain embodiments, the subject is further administered at least one additional agent or therapy useful for treating, preventing, and/or ameliorating cancer in a subject. In certain embodiments, the additional agent or therapy is administered concurrent with, prior to, or subsequent to the TLR4 inhibitor. In certain embodiments, the additional agent or therapy is selected from the group consisting of a systemic chemotherapy, a topical chemotherapy, an immunotherapy, an antibody-drug conjugate (ADC), an antibody, an immunotoxin, an interferon, and any combination thereof.

In certain embodiments, the subject is a mammal. In certain embodiments, the mammal is a human.

### Pharmaceutical Compositions

In another aspect, the present disclosure provides a pharmaceutical composition comprising a TLR4 inhibitor and at least one pharmaceutically acceptable carrier.

Such a pharmaceutical composition may consist of at least one composition of the invention, in a form suitable for administration to a subject, or the pharmaceutical composition may comprise at least one composition, and one or more pharmaceutically acceptable carriers, one or more additional ingredients, or any combinations of these. At least one composition of the invention may be present in the pharmaceutical composition in the form of a physiologically acceptable salt, such as in combination with a physiologically acceptable cation or anion, as is well known in the art.

In certain embodiments, the pharmaceutical compositions useful for practicing the method of the invention may be administered to deliver a dose of between 1 ng/kg/day and 100 mg/kg/day. In other embodiments, the pharmaceutical compositions useful for practicing the invention may be administered to deliver a dose of between 1 ng/kg/day and 1,000 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for nasal, inhalational, oral, rectal, vaginal, pleural, peritoneal, parenteral, topical, transdermal, pulmonary, intranasal, buccal, ophthalmic, epidural, intrathecal, intravenous, or another route of administration. A composition useful within the methods of the invention may be directly administered to the brain, the brainstem, or any other part of the central nervous system of a mammal or bird. Other contemplated formulations include projected nanoparticles, microspheres, liposomal preparations, coated particles, polymer conjugates, resealed erythrocytes containing the active ingredient, and immunologically based formulations. In certain embodiments, the compositions of the invention are part of a pharmaceutical matrix, which allows for manipulation of insoluble materials and improvement of the bioavailability thereof, development of controlled or sustained release products, and generation of homogeneous compositions. By way of example, a pharmaceutical matrix may be prepared using hot melt extrusion, solid solutions, solid dispersions, size reduction technologies, molecular complexes (e.g., cyclodextrins, and others), microparticulate, and particle and formulation coating processes. Amorphous or crystalline phases may be used in such processes. The route(s) of administration will be readily apparent to the skilled artisan and will depend upon any number of factors including the type and severity of the disease being treated, the type and age of the patient being treated, and the like.

The formulations of the pharmaceutical compositions described herein may be prepared by any method known or hereafter developed in the art of pharmacology and pharmaceutics. In general, such preparatory methods include the step of bringing the active ingredient into association with a carrier or one or more other accessory ingredients, and then, if necessary or desirable, shaping or packaging the product into a desired single-dose or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In certain embodiments, the compositions of the invention are formulated using one or more pharmaceutically acceptable excipients or carriers. In certain embodiments, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, ethanol, recombinant human albumin (e.g., RECOMBUMIN®), solubilized gelatins (e.g., GELOFUSINE®), and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), recombinant human albumin, solubilized gelatins, suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, isotonic agents, for example, sugars, sodium chloride, or polyalcohols such as mannitol and sorbitol, are included in the composition. Prolonged absorption of the injectable compositions may be brought about by including in the composition an agent that delays absorption, for example, aluminum monostearate or gelatin.

Formulations may be employed in admixtures with conventional excipients, i.e., pharmaceutically acceptable organic or inorganic carrier substances suitable for oral, parenteral, nasal, inhalational, intravenous, subcutaneous, transdermal enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring, and/or fragrance-conferring substances and the like. They may also be combined where desired with other active agents, e.g., other analgesic, anxiolytics or hypnotic agents. As used herein, “additional ingredients” include, but are not limited to, one or more ingredients that may be used as a pharmaceutical carrier.

The composition of the invention may comprise a preservative from about 0.005% to 2.0% by total weight of the composition. The preservative is used to prevent spoilage in the case of exposure to contaminants in the environment. Examples of preservatives useful in accordance with the invention include but are not limited to those selected from the group consisting of benzyl alcohol, sorbic acid, parabens, imidurea and any combinations thereof. One such preservative is a combination of about 0.5% to 2.0% benzyl alcohol and 0.05-0.5% sorbic acid. The composition may include an antioxidant and a chelating agent that inhibit the degradation of the compound. Antioxidants for some compounds are BHT, BHA, alpha-tocopherol and ascorbic acid in the exemplary range of about 0.01% to 0.3%, or BHT in the range of 0.03% to 0.1% by weight by total weight of the composition. The chelating agent may be present in an amount of from 0.01% to 0.5% by weight by total weight of the composition.

Exemplary chelating agents include edetate salts (e.g., disodium edetate) and citric acid in the weight range of about 0.01% to 0.20%, or in the range of 0.02% to 0.10% by weight by total weight of the composition. The chelating agent is useful for chelating metal ions in the composition that may be detrimental to the shelf life of the formulation. While BHT and disodium edetate are exemplary antioxidant and chelating agent, respectively, for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethyl cellulose.

Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, acacia, and ionic or non-ionic surfactants. Known preservatives include, but are not limited to, methyl, ethyl, or n-propyl para-hydroxybenzoates, ascorbic acid, and sorbic acid.

Known sweetening agents include, for example, glycerol, propylene glycol, sorbitol, sucrose, and saccharin.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying. Methods for mixing components include physical milling, the use of pellets in solid and suspension formulations and mixing in a transdermal patch, as known to those skilled in the art.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily, or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg, or less than about 200 mg, or less than about 50 mg.

Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXAMPLES

Various embodiments of the present application can be better understood by reference to the following Examples which are offered by way of illustration. The scope of the present application is not limited to the Examples given herein.

### Example 1: Role of TLR4 Signaling in Cutaneous T-cell Lymphoma

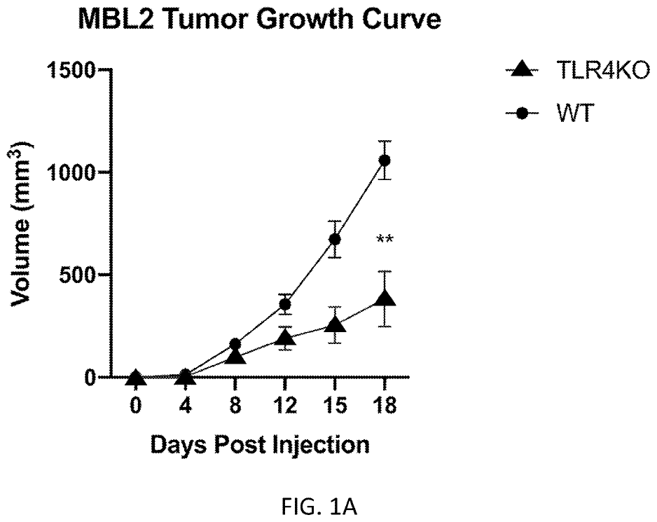

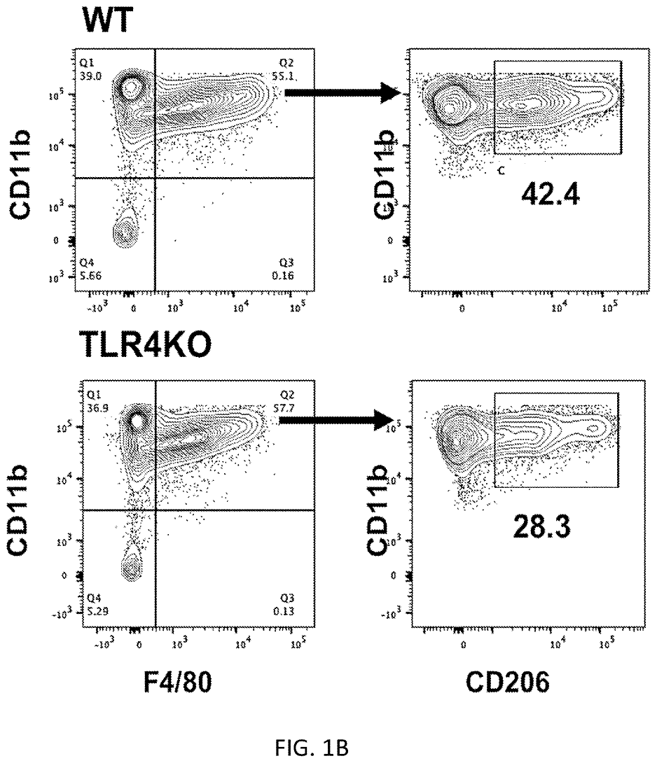

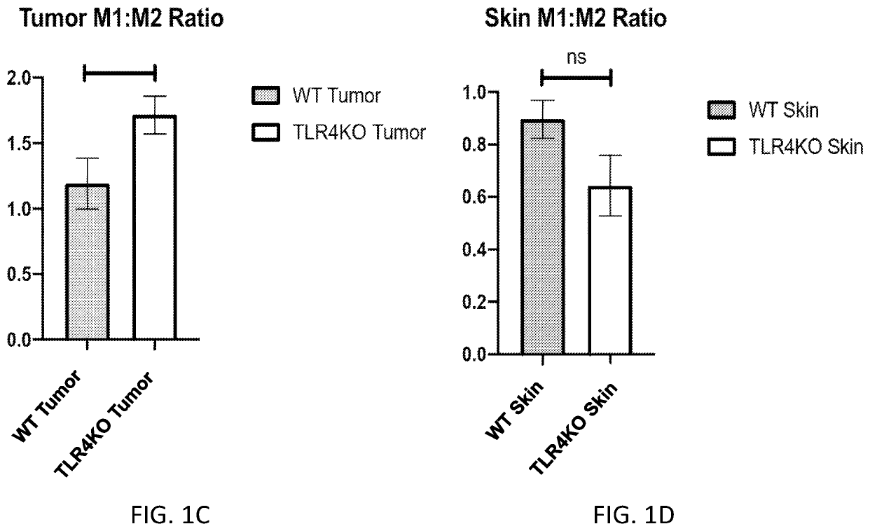

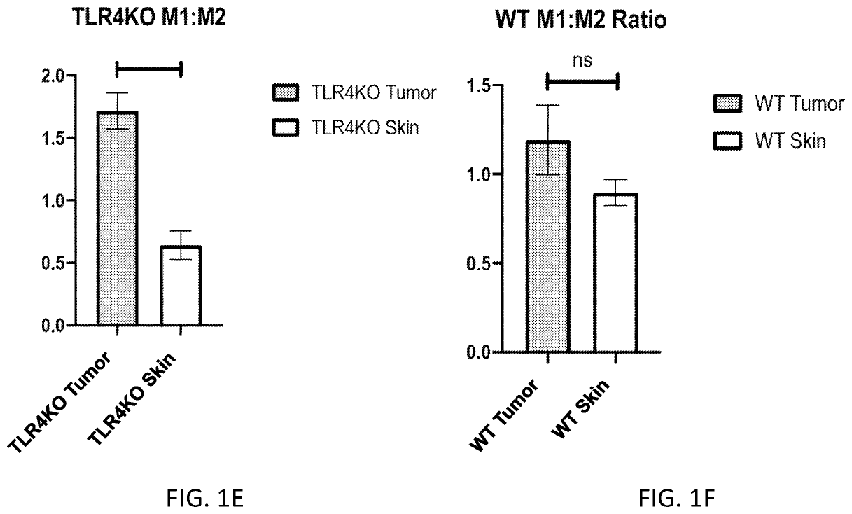

Initial studies examined the contribution of TLR4 expression on in situ cutaneous T-cell lymphoma (CTCL) tumor growth. Growth rates for MBL2 tumors established in TLR4-/-host animals were determined and found that CTCL tumor growth is attenuated in TLR4 deficient mice (FIG. 1A).

Follow-up studies then sought to evaluate the impact of loss of TLR4 expression on CTCL-infiltrating macrophages. To examine the impact of TLR4 on CTCL-infiltrating macrophages, tumors were generated in TLR4 deficient mice. At all experimental endpoints, tumors and skin of tumor bearing mice were harvested and homogenized to perform flow cytometric analyses. M2 macrophages in tumors were assayed by staining and gating for the CD11b+F4/80+CD206+ population using the gating strategy demonstrated in FIG. 1B, while M1 macrophages were identified as CD11b+F4/80+CD206−. Overall, the M1/M2 macrophage ratio was significantly higher in tumors generated in TLR4 deficient mice compared to wild type (FIG. 1C), but this difference was not detected in the skin of these mice (FIG. 1D). Furthermore, the M1/M2 ratio was significantly higher in tumors compared to skin in TLR4 deficient mice (FIG. 1E), while these ratios did not differ significantly in wild-type mice (FIG. 1F). Overall, and without wishing to be bound by theory, these results suggest a dynamic shift from tumor infiltrating M2 macrophage towards MI macrophage phenotype in TLR4 deficient mice. These data further suggest that the increased presence of pro-inflammatory M1 macrophages along with decreased immunosuppressive macrophages may be associated with the decreased tumor burden seen in TLR4KO mice by enhancing endogenous anti-tumor immune responses.

### Example 2: The Contribution of Pharmaceutical TLR4 Inhibition on In Situ CTCL Tumor Growth

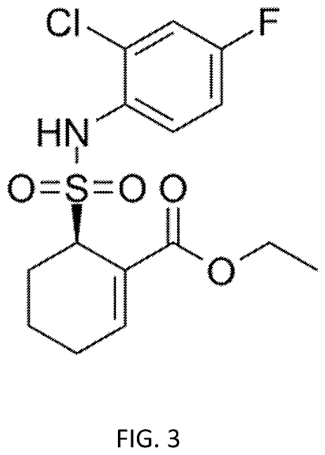

Having demonstrated that loss of TLR4 function by genetic knockout could slow the growth of tumors in vivo, and that the delay was associated by a change in the population balance of tumor-infiltrating macrophages from tolerance-associated M2 macrophages to pro-inflammatory MI macrophages, studies then sought to determine whether the use of an inhibitor of TLR4 could result similar effects. For these studies, the inhibitor TAK-242 was selected (See structure in FIG. 3). Also known as resatorvid, this small molecule selectively binds to the intracellular domain of TLR4 where it interferes with TLR4 interaction with adaptor signaling molecules.

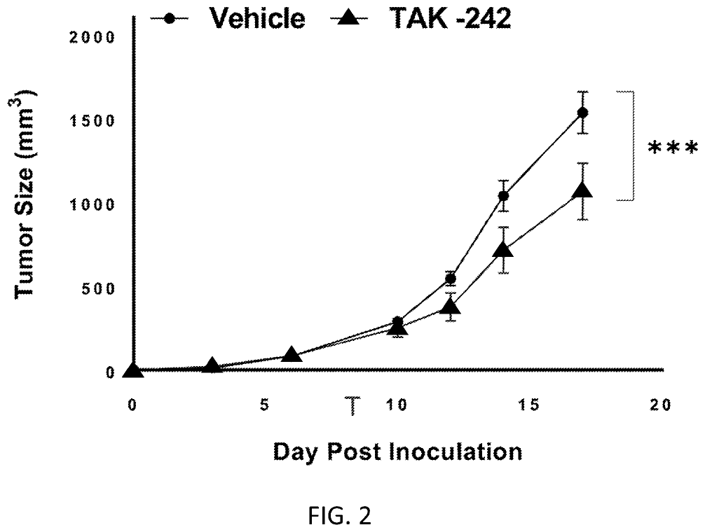

For these studies, 2×10⁶ MBL2 cells were injected subcutaneously into the flanks of C57BL/6 mice. When tumors reached an average volume of 85 mm, mice were allocated to vehicle (n=5) or TAK-242 (n=5) treatment groups. All mice were treated daily with intraperitoneal injections of vehicle (95% saline 5% DMSO) or 3 mg/kg of TAK-242. Tumor volumes were monitored three times per week. First, the growth rates for MBL2 tumors in hosts treated TLR4 inhibitor TAK-242 and found that CTCL tumor growth is attenuated in mice treated with TLR4 inhibitor (FIG. 2). Tumor sizes are shown as average and SEM per group.

### Enumerated Embodiments

The following exemplary embodiments are provided, the numbering of which is not to be construed as designating levels of importance.

Embodiment 1 provides a method for treating cancer in a subject in need thereof, comprising administering to the subject an effective amount of an inhibitor of Toll-Like Receptor 4 (TLR4), thereby treating the cancer;
wherein treatment with the TLR4 inhibitor induces a pro-inflammatory phenotype in at least one tumor-associated immune cell.

Embodiment 2 provides the method of embodiment 1, wherein the TLR4 inhibitor is TAK-242.

Embodiment 3 provides the method of embodiment 1, wherein the tumor-associated immune cell is a monocyte selected from the group consisting of a macrophage, a dendritic cell, or any combination thereof.

Embodiment 4 provides the method of embodiment 1, wherein the tumor-associated immune cell is a macrophage.

Embodiment 5 provides the method of embodiment 4, wherein the macrophage is a M1 macrophage.

Embodiment 6 provides the method of embodiment 1, wherein the pro-inflammatory phenotype is a change in the ratio of M1 to M2 macrophages.

Embodiment 7 provides the method of embodiment 1, wherein the cancer is a lymphoma.

Embodiment 8 provides the method of embodiment 7, wherein the lymphoma is cutaneous T-cell lymphoma (CTCL).

Embodiment 9 provides the method of embodiment 8, wherein the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

Embodiment 10 provides the method of embodiment 1, wherein the TLR4 inhibitor is delivered systemically.

Embodiment 11 provides the method of embodiment 1, wherein the TLR4 inhibitor is delivered topically.

Embodiment 12 provides a composition for treating cancer, comprising an effective amount of a TLR4 inhibitor and a pharmaceutically acceptable carrier or excipient.

Embodiment 13 provides the composition of embodiment 12, wherein the TLR4 inhibitor is TAK-242.

Embodiment 14 provides the composition of embodiment 12, wherein the cancer is a lymphoma.

Embodiment 15 provides the composition of embodiment 14, wherein the lymphoma is cutaneous T-cell lymphoma.

Embodiment 16 provides the composition of embodiment 15, wherein the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

Embodiment 17 provides the method of embodiment 1, further comprising administering to the subject at least one other therapeutic agent concurrent with, prior to, or subsequent to the TLR4 inhibitor.

Embodiment 18 provides the method of embodiment 18, wherein the therapeutic agent is selected from the group consisting of a systemic chemotherapy, a topical chemotherapy, an immunotherapy, an antibody-drug conjugate (ADC), an antibody, an immunotoxin, an interferon, and any combination thereof.

### Other Embodiments

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

What is claimed is:

1. A method for treating cancer in a subject in need thereof, comprising administering to the subject an effective amount of an inhibitor of Toll-Like Receptor 4 (TLR4), thereby treating the cancer;
wherein treatment with the TLR4 inhibitor induces a pro-inflammatory phenotype in at least one tumor-associated immune cell.

2. The method of claim 1, wherein the TLR4 inhibitor is TAK-242.

3. The method of claim 1, wherein the tumor-associated immune cell is a monocyte selected from the group consisting of a macrophage, a dendritic cell, or any combination thereof.

4. The method of claim 1, wherein the tumor-associated immune cell is a macrophage.

5. The method of claim 4, wherein the macrophage is a MI macrophage.

6. The method of claim 1, wherein the pro-inflammatory phenotype is a change in the ratio of M1 to M2 macrophages.

7. The method of claim 1, wherein the cancer is a lymphoma.

8. The method of claim 7, wherein the lymphoma is cutaneous T-cell lymphoma (CTCL).

9. The method of claim 8, wherein the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.

10. The method of claim 1, wherein the TLR4 inhibitor is delivered systemically.

11. The method of claim 1, wherein the TLR4 inhibitor is delivered topically.

12. The method of claim 1, further comprising administering to the subject at least one other therapeutic agent concurrent with, prior to, or subsequent to the TLR4 inhibitor.

13. The method of claim 12, wherein the therapeutic agent is selected from the group consisting of a systemic chemotherapy, a topical chemotherapy, an immunotherapy, an antibody-drug conjugate (ADC), an antibody, an immunotoxin, an interferon, and any combination thereof.

14. A composition for treating cancer, comprising an effective amount of a TLR4 inhibitor and a pharmaceutically acceptable carrier or excipient.

15. The composition of claim 14, wherein the TLR4 inhibitor is TAK-242.

16. The composition of claim 14, wherein the cancer is a lymphoma.

17. The composition of claim 16, wherein the lymphoma is cutaneous T-cell lymphoma.

18. The composition of claim 17, wherein the cutaneous T-cell lymphoma is selected from the group consisting of mycosis fungoides, Sezary syndrome, primary cutaneous CD30⁺ lymphoproliferative disorder, and primary cutaneous gamma-delta T-cell lymphoma.
